(19) United States
(12) Patent Application Publication
(10) Pub. No.: US 2011/0318398 A1
(43) Pub. Date: Dec. 29, 2011

(54) COSMETIC COMPOSITION COMPRISING MOLECULAR ENCAPSULATED FERMENTED EXTRACT OF RHUS JAVANICA L. AS AN ACTIVE INGREDIENT

(75) Inventors: Seong Kweon Lee, Seoul (KR); Jueun Song, Seoul (KR); Young Dae Park, Seoul (KR); Sung-Hee Cho, Seoul (KR)

(73) Assignee: KOREA NATURAL SCIENCE CO., LTD., Seoul (KR)

(21) Appl. No.: 13/114,933

(22) Filed: May 24, 2011

(30) Foreign Application Priority Data
May 25, 2010 (KR) 10-2010-0048784

(51) Int. Cl. A61K 8/11; A61Q 19/00; A61Q 19/02; A61K 8/97

(52) U.S. Cl. 424/401; 424/62; 424/769

(57) ABSTRACT

The present invention relates to a cosmetic composition comprising a composite prepared by molecularly encapsulating a fermented sumac (Rhus javanica L.) extract with a cyclodextrin derivative as an active ingredient. More particularly, the present invention relates to a skin-whitening or antioxidant composition comprising a composite prepared by molecularly encapsulating a fermented sumac (Rhus javanica L.) extract with hydroxypropyl-β-cyclodextrin as an active ingredient. The composition of the present invention exhibits better skin-whitening and antioxidant effect than the existing cosmetic composition comprising 4-n-butylresorcinol, while having better stability. Further, since it is originated from natural materials with much less skin irritation, it can be effectively used to prepare functional skin-whitening and antioxidant cosmetic products.

### TECHNICAL FIELD

The present invention relates to a cosmetic composition comprising a composite prepared by molecularly encapsulating a fermented sumac (Rhus javanica L.) extract with a cyclodextrin derivative as an active ingredient. More particularly, the present invention relates to a skin-whitening or antioxidant composition comprising a composite prepared by molecularly encapsulating a fermented sumac (Rhus javanica L.) extract with hydroxypropyl-β-cyclodextrin as an active ingredient.

### BACKGROUND

Prolonged exposure to oxidative stresses from harmful environments including air pollution, UV, stress or diseases leads to increased level of radicals in the body and damage to collagen, elastin, hyaluronic acid, etc. constituting the connective tissues of the dermis, thereby resulting in skin wrinkling. Also, it may induce such diseases as dermatitis, acne or skin cancer by oxidizing the lipids of cell membranes and thereby leading to cell damage. In addition, the radicals may cause liver spots, freckles and wrinkles through production of melanin. Formerly, ascorbic acid, α-tocopherol, SOD, etc. have been used in cosmetics or medications as free radical scavengers to prevent wrinkles and other skin diseases. However, they are expensive and are not chemically stable when mixed. For these reasons, development of safe and stable substances exhibiting excellent free radical-scavenging effect is an important issue not only in the medicine and food industries but also in the cosmetics industry.

Hyperpigmentation of the skin may be caused by several factors, including hormone changes following skin inflammations, genetic disease, UV radiation, or the like. The main cause is abnormality in the synthesis and distribution of melanin.

Melanin plays an important role of removing oxyradicals and thereby protecting the skin from damage. Therefore, abundance of melanin means that there is an effective defense system for protecting the skin from physically and chemically toxic substances. The production of melanin is accomplished in melanocytes through conversion of tyrosine to dopaquinone catalyzed by tyrosinase, followed by catalytic and spontaneous oxidation reactions. Therefore, methods for suppressing melanin production to prevent skin darkening are as follows.

Firstly, UV may be blocked to eliminate the main cause of melanin production. For this, a light scattering agent or a light blocking agent may be included in a cosmetic composition.

Secondly, the synthesis of core carbohydrates necessary for tyrosinase to be active, such as glucosamine, may be inhibited to suppress the melanin production.

Thirdly, kojic acid or arbutin may be used to inhibit tyrosinase and thus to prevent melanin production.

Fourthly, substances specifically toxic to melanin-producing melanocytes, such as hydroquinone, may be used.

In addition, once-produced melanin may be reduced.

As described, ascorbic acid, kojic acid, arbutin, hydroquinone, and natural plant extracts have been used as skin-lightening agents for suppressing the production of melanin. However, although kojic acid, arbutin or 4-n-butylresorcinol (lucinol) provides good skin-whitening effect, they have safety problems due to irritation.

Particularly, 4-n-butylresorcinol is easily browned due to poor stability and causes skin irritation when used at high concentration.

In order to solve the problem of 4-n-butylresorcinol, Korean Patent No. 10-751883 uses γ-linolenic acid to increase stability of 4-n-butylresorcinol (lucinol) through liposomization and reduce skin irritation (hereinafter, referred to as “whitenol”). However, the effect is not sufficient.

Accordingly, development of a cosmetic composition comprising 4-n-butylresorcinol providing excellent whitening effect and high stability without skin irritation is required.

### SUMMARY

The inventors of the present invention have studied for a cosmetic composition with improved water solubility, stability and whitening effect and without skin irritation. They have found out that a fermented sumac (Rhus javanica L.) extract has improved skin-whitening and antioxidant effect, and water solubility and stability can be improved through molecular encapsulation, thereby completing the invention.

Accordingly, the present invention is directed to providing a whitening cosmetic composition comprising a composite prepared by molecularly encapsulating a fermented sumac (Rhus javanica L.) extract with a cyclodextrin derivative as an active ingredient.

The present invention is also directed to providing an antioxidant cosmetic composition comprising a composite prepared by molecularly encapsulating a fermented sumac (Rhus javanica L.) extract with a cyclodextrin derivative as an active ingredient.

In one general aspect, the present invention provides a whitening cosmetic composition comprising a composite prepared by molecularly encapsulating a fermented sumac (Rhus javanica L.) extract with a cyclodextrin derivative as an active ingredient.

In another general aspect, the present invention provides an antioxidant cosmetic composition comprising a composite prepared by molecularly encapsulating a fermented sumac (Rhus javanica L.) extract with a cyclodextrin derivative as an active ingredient.

### BRIEF DESCRIPTION OF THE DRAWINGS

FIG. 1 shows a photograph of a whitening cosmetic composition according to the present invention, comprising a composite prepared by molecularly encapsulating a fermented sumac (Rhus javanica L.) extract with a cyclodextrin derivative as an active ingredient fermented sumac (Rhus javanica L.) extract, dissolved in water;

FIG. 2 schematically shows a structure of a composite according to the present invention, prepared by molecularly encapsulating a fermented sumac (Rhus javanica L.) extract with a cyclodextrin derivative;

FIG. 3 shows a result of comparing inhibitory effect against tyrosinase activity of a composition according to the present invention with that of whitenol (The abscissa represents the concentration of the composition of the present invention or whitenol as control.);

FIG. 4 shows a result of measuring tyrosinase activity inhibition effect of arbutin at different concentrations;

FIG. 5 shows a result of comparing free radical scavenging effect of a composition according to the present invention with that of whitenol (The abscissa represents the concentration of the composition of the present invention or whitenol as control.).

FIG. 6 shows a result of evaluating stability of a composition according to the present invention (FIG. 6A shows a result after keeping at room temperature for 30 days, and FIG. 6B shows a result after keeping at 45° C. for 30 days. (a): cosmetics containing the composition of the present invention, (b): cosmetics containing 4-n-butylresorcinol).

FIG. 7 shows a result of evaluating skin irritation of a composition according to the present invention ((a): the part that has been contacted with cosmetics containing 4-n-butylresorcinol, (b): the part that has been contacted with cosmetics containing the composition of the present invention).

### DETAILED DESCRIPTION OF EMBODIMENTS

Hereinafter, exemplary embodiments will be described in detail with reference to the accompanying drawings.

The present invention provides a whitening cosmetic composition or an antioxidant cosmetic composition comprising a composite prepared by molecularly encapsulated a fermented sumac (Rhus javanica L.) extract with a cyclodextrin derivative as an active ingredient.

The cosmetic composition of the present invention comprises a composite prepared by molecularly encapsulating a sumac (Rhus javanica L.) extract fermented with lactic acid bacteria and then purified as an active ingredient.

Sumac (Rhus javanica L.) is a small deciduous shrub belonging to the family Anacardiaceae. It is reported that the bark extract of sumac (Rhus javanica L.) has antioxidant effect (Youn Jae Lee et al., 1993, Food Sci. Biotechnol. 25(6): 677-682).

The sumac (Rhus javanica L.) extract can be prepared by a known solvent extraction method. As an extraction solvent, for example, water, a C₁-C₆ alcohol such as ethanol and methanol, an organic solvent such as acetone, ethyl acetate, n-hexane, diethyl ether and benzene, or a mixture thereof may be used. Specifically, a solvent selected from a group consisting of water, a C₁-C₆ alcohol and a mixture thereof may be used for the extraction.

Most specifically, the extraction solvent may be ethanol. The extraction may be performed by any common extraction methods, including cold extraction and hot extraction. When ethanol is used for the extraction, the proportion of sumac (Rhus javanica L.) to ethanol is not particularly limited. For example, ethanol may be added to sumac (Rhus javanica L.) in an amount of 3 to 20 times based on weight. Specifically, water may be added in an amount of 8 to 12 times based on sumac (Rhus javanica L.) in order to improve extraction efficiency. Extraction temperature is not particularly limited as long as the destruction of the extracted components can be avoided. Specifically, the extraction temperature may be 4° C. to 120° C. Most specifically, it may be 60° C. to 100° C. Extraction time may be different depending on the extraction temperature and the extraction solvent. Typically, the extraction is carried out for 0.5 to 24 hours, specifically 0.5 to 3 hours.

The part of sumac (Rhus javanica L.) to be extracted may be anything, including bark, leaf and root. Specifically, it may be bark. The sumac (Rhus javanica L.) bark may be extracted after washing and then with or without drying. The drying may be performed by any method, including sun drying, shade drying, hot-air drying, and air drying. Furthermore, sumac (Rhus javanica L.) may be grounded to enhance the extraction efficiency.

The extract of the present invention includes not only one extracted using the above-described extraction solvent but also one purified according to a common method. For example, fractions obtained through a variety of further purification processes, including separation of precipitates using filter paper, separation using an ultrafiltration membrane with a predetermined molecular weight cutoff value, separation by various chromatography techniques (based on size, charge, hydrophobicity or affinity), bleaching, etc., are included in the scope of the sumac (Rhus javanica L.) extract of the present invention. Specifically, the extract of the present invention may be further separated through filter paper for separation of precipitates and concentrated. The concentration may be performed by using a known concentrating apparatus or by heating in a water bath at 100° C. or lower temperature.

The lactic acid bacteria used for fermentation in the present invention refer to the microorganisms that produce lactic acid as the final metabolic product by fermenting carbohydrates. They are commonly utilized in the preparation of fermented food products including kimchi and yogurt. The lactic acid bacteria may be one or more selected from those belonging to the genera Enterococcus, Lactobacillus, Lactococcus, Leuconostoc, Streptococcus and Weisella. The lactic acid bacteria may be used in admixture with the bacteria belonging to the genera Bifidobacterium, Lactobacillus or Lactococcus. Specifically, Lactobacillus may be used. More specifically, the lactic acid bacteria of the present invention may be Lactobacillus casei.

The fermentation may be performed by a known fermentation method. Any fermentation medium to which the sumac (Rhus javanica L.) extract is added may be used. Specifically, a medium containing concentrated sumac (Rhus javanica L.) extract as well as glucose may be used.

The fermented extract may comprise 4-n-butylresorcinol. The inventors of the present invention found out that, although 4-n-butylresorcinol is not found in the sumac (Rhus javanica L.) extract, it exists in the fermented extract.

4-n-Butylresorcinol is known as a synthetic compound for skin whitening. When used in high concentration, it causes skin irritation and has decreased stability. It has never been reported that the fermented sumac (Rhus javanica L.) extract contains 4-n-butylresorcinol.

The fermented extract of the present invention includes one purified after the fermentation. For example, fractions obtained through a variety of further purification processes, including separation of precipitates using filter paper, separation using an ultrafiltration membrane with a predetermined molecular weight cutoff value, separation by various chromatography techniques (based on size, charge, hydrophobicity or affinity), separation by cooling crystallization, bleaching, etc., are included in the scope of the fermented sumac (Rhus javanica L.) extract of The present invention. Specifically, the fermented extract of the present invention may be further separated through filter paper for removal of the fermenting bacteria and concentrated. The concentration may be performed by using a known concentrating apparatus or by heating in a water bath at 100° C. or lower temperature. More specifically, the fermented extract of the present invention may be further purified by cooling crystallization. For the purification, any known solvent capable of dissolving 4-n-butylresorcinol may be used. Specifically, a mixture of water and ethanol may be used. Following the purification, the composition of the present invention has an increased 4-n-butylresorcinol content. Specifically, the fermented sumac (Rhus javanica L.) extract of the present invention may comprise 4-n-butylresorcinol in an amount of 20 to 99 wt % based on the total weight of the fermented sumac extract.

α-, β- and γ-cyclodextrins are well known as cage molecules capable of encapsulating hydrophobic molecules. However, cyclodextrins do not have good solubility in aqueous solutions. To solve this problem, cyclodextrin derivatives having various substituents attached are used. As new functional biomaterials, cyclodextrin derivatives have a doughnut-shaped ring structure composed of 6-8 glucose units. The cavity in the molecule is hydrophobic whereas the exterior is hydrophilic. Accordingly, since the cyclodextrin derivatives can stabilize various unstable hydrophobic compounds by encapsulating them in the hydrophobic cavity, i.e. via molecular encapsulation, they are widely utilized in medicine, food and other applications.

Specifically, the cyclodextrin derivative for molecular encapsulation according to The present invention may be 2,6-dimethyl-β-cyclodextrin, hydroxyethyl-β-cyclodextrin, hydroxyethyl-γ-cyclodextrin, hydroxypropyl-β-cyclodextrin, hydroxypropyl-γ-cyclodextrin, (2-carboxymethoxy)propyl-β-cyclodextrin or sulfobutylether-7-β-cyclodextrin. More specifically, it may be hydroxypropyl-β-cyclodextrin.

The cosmetic composition of the present invention provides a whitening effect by inhibiting the activity of tyrosinase involved in melanin production. The effect is better that that of the existing cosmetic compositions comprising 4-n-butylresorcinol. Also, the cosmetic composition of the present invention provides an antioxidant effect of scavenging free radicals. The effect is better that that of the existing cosmetic compositions comprising 4-n-butylresorcinol. In addition, the cosmetic composition of the present invention exhibits a remarkably better stability as well as less skin irritation as compared to the existing cosmetic compositions comprising 4-n-butylresorcinol.

These advantageous effects of the present invention are demonstrated through the examples.

In an example according to the present invention, dried sumac (Rhus javanica L.) bark was added to ethanol at a proportion of 1:10 and extracted for 1 hour at 80° C. Then, a concentrated sumac (Rhus javanica L.) extract obtained by evaporating ethanol was fermented with Lactobacillus casei for 72 hours to prepare a fermented sumac (Rhus javanica L.) extract according to the present invention. The prepared fermented sumac (Rhus javanica L.) extract was purified by diluting with ethanol, heating and then cooling slowly. The purified extract was fractionated using a column.

Analysis of the concentrate prior to the fermentation and the fermented product and fractions following the fermentation revealed that the concentrate prior to the fermentation contains no 4-n-butylresorcinol but the fermented product contains 4-n-butylresorcinol. The fractions showed remarkably higher concentrations (see Examples 1 and 2).

In another example according to the present invention, the fermented sumac (Rhus javanica L.) extract was molecularly encapsulated. After completely dissolving hydroxypropyl-6-cyclodextrin in water at 60° C., the fermented sumac (Rhus javanica L.) extract was added and then slowly cooled after mixing (see Example 3).

In another example according to the present invention, the inhibitory effect of the composition of the present invention against tyrosinase activity was measured after adding the fermented sumac (Rhus javanica L.) extract to a buffer solution together with tyrosine and tyrosinase. As a result, it was confirmed that the composition of the present invention exhibits much better tyrosinase activity inhibition effect than whitenol (liposomized 4-n-butylresorcinol) as control (see Example 4).

In another example according to the present invention, the antioxidant effect of scavenging free radicals of the composition of the present invention was evaluated by measuring the change in absorbance of a 1,1-diphenyl-2-picrylhydrazyl (DPPH) solution by the fermented sumac (Rhus javanica L.) extract. As a result, it was confirmed that the composition of the present invention exhibits much better antioxidant effect than whitenol as control (see Example 5).

In another example according to the present invention, the stability of the composition of the present invention was tested. After preparing nourishing creams using the composition of the present invention comprising a composite prepared by molecularly encapsulating the fermented sumac (Rhus javanica L.) extract with a cyclodextrin derivative or using 4-n-butylresorcinol, change in color was observed while keeping them at room temperature or at 45° C. for 30 days.

The nourishing cream containing 4-n-butylresorcinol showed severe color change after being kept at 45° C. for 30 days, but the nourishing cream containing the composition of the present invention showed no color change after being kept at room temperature or at 45° C. Thus, it was confirmed that the cosmetic composition of the present invention has much better stability than the existing cosmetic composition comprising 4-n-butylresorcinol (see Example 6).

In another example according to the present invention, skin irritation of the composition of the present invention was tested. After preparing nourishing creams using the composition of the present invention comprising a composite prepared by molecularly encapsulating the fermented sumac (Rhus javanica L.) extract with a cyclodextrin derivative or using 4-n-butylresorcinol, patch test was carried out for 15 subjects.

As a result, the part that had been contacted with the cosmetics containing the composition of the present invention showed no abnormalities in all the subjects after 48 hours, whereas the part that had been contacted with the cosmetics containing 4-n-butylresorcinol showed skin irritation and erythema in 7 out of the 15 subjects. Thus, it was confirmed that the cosmetic composition of the present invention exhibits much less skin irritation than the existing cosmetic composition comprising 4-n-butylresorcinol (see Example 7).

Therefore, since the composite prepared by molecularly encapsulating the fermented sumac (Rhus javanica L.) extract with the cyclodextrin derivative exhibits excellent skin-whitening effect and antioxidant effect, it may be effectively used as an active ingredient in a whitening or antioxidant cosmetic composition.

The cosmetic composition of the present invention comprises the composite prepared by molecularly encapsulating the fermented sumac (Rhus javanica L.) extract with the cyclodextrin derivative as an active ingredient and may be prepared into a base cosmetic composition (e.g., lotion, cream, essence, cleanser such as cleansing foam or cleansing water, pack, body oil, etc.), a coloring cosmetic composition (e.g., foundation, lipstick, mascara, makeup base, etc.), a hair product composition (e.g., shampoo, rinse, hair conditioner, hair gel, etc.), soap or the like, along with a dermatologically acceptable excipient.

The excipient may include, for example, a skin emollient, a skin penetration enhancer, a coloring agent, an aromatic, an emulsifier, a thickener and a solvent, without being limited thereto. Also, a fragrance, a pigment, a sterilizer, an antioxidant, an antiseptic, a moisturizer, etc. may be further included, and a viscosity enhancer, minerals, synthetic polymer materials, etc. may be included to improve physical properties. For example, the cosmetic composition of the present invention may be prepared easily into a facial cleanser or soap by adding the composite prepared by molecularly encapsulating the fermented sumac (Rhus javanica L.) extract with the cyclodextrin derivative to a commonly used facial cleanser or soap base. Also, a cream may be prepared by adding the composite prepared by molecularly encapsulating the fermented sumac (Rhus javanica L.) extract with the cyclodextrin derivative to a commonly used oil-in-water (O/W) type cream base. A fragrance, a chelating agent, a pigment, an antioxidant, an antiseptic, and synthetic or natural substances for improvement of physical properties such as proteins, minerals and vitamins may be further added.

The content of the composite prepared by molecularly encapsulating the fermented sumac (Rhus javanica L.) extract with the cyclodextrin derivative in the cosmetic composition of the present invention is not particularly limited. Specifically, it may be included in an amount of 0.001 to 10 wt %, more preferably 0.01 to 5 wt %, based on the total weight of the composition. When the content is less than 0.001 wt %, the desired whitening and antioxidant effect may not be achieved. And, even when it exceeds 10 wt %, there is no further improvement.

### EXAMPLES

The examples and experiments will now be described. The following examples and experiments are for illustrative purposes only and not intended to limit the scope of this disclosure.

### Example 1

Preparation of Fermented Sumac (Rhus javanica L.) Extract

100 g of dried sumac (Rhus javanica L.) bark (purchased from Nonglim Herb in the Gyeongdong Market) was added to 1 L of ethanol. After boiling at 80° C. for 1 hour under reflux followed by filtration, ethanol was evaporated from the resultant extract to finally obtain 18 g of a concentrated sumac (Rhus javanica L.) extract.

To 1 L of a medium containing 18 g of the concentrated sumac (Rhus javanica L.) extract as well as 9 g of glucose, 100 g of a fermentation broth (a broth prepared by incubating Lactobacillus casei PM1 (KCCM 10766P) overnight in a medium containing 10.0 g of Proteose Peptone No. 3, 10.0 g of beef extract, 5.0 g of yeast extract, 20.0 g of dextrose, 1.0 g of Polysorbate 80, 2.0 g of ammonium citrate, 5.0 g of sodium acetate, 0.1 g of magnesium sulfate, 0.05 g of manganese sulfate and 20.0 g of dipotassium phosphate) was added. Then, fermentation was carried out for 72 hours at 37.5° C., while maintaining pH at 6.5.

The resulting fermentation solution was filtered through 0.2-μm filter to remove the bacteria and impurities. The filtrate was evaporated and the solid contents were analyzed.

As seen from [Table 1], 4-n-butylresorcinol was not detected in the sumac (Rhus javanica L.) extract, but it was detected when the sumac (Rhus javanica L.) extract was fermented with Lactobacillus casei PM1 (KCCM 10766P).

### Example 2

Purification of Fermented Sumac (Rhus javanica L.) Extract

The fermentation solution prepared in <Example 1> was filtered through 0.2-μm filter to remove the bacteria and impurities. After evaporating more than half of water from 1 L of the filtrate using a rotary vacuum evaporator, 0.5 L of ethanol was added and mixed to obtain a turbid solution. After heating to 70° C. for crystallization, when the solution turned clear, it was cooled to 5° C. at a constant rate of −0.2° C./min. During this procedure, a considerable amount of fine crystals were precipitated. After the cooling, the crystals were recovered by filtration and completely dissolved in 10 to 15 times volume equivalents of a mixture solvent (water:ethanol=1:1). Then, the solution was cooled to 5° C. at a constant rate of −0.2° C./min. This procedure was repeated 2-3 times to obtain a highly pure product.

The recovered crystals were dissolved in 0.5 L of ethanol and eluted using a column packed with 100 g of the synthetic filler HP-20 (Mitsubishi Chemical Corporation, Japan). First, after eluting 0.5 L of 100% water to remove water-soluble components, fractionation was carried out while increasing the amount of ethanol in the eluent from 5% to 50%.

As seen from [Table 1], the ethanol 25% fraction obtained from the purification had the highest 4-n-butylresorcinol content. The content of the active ingredient was analyzed by high-performance liquid chromatography (HPLC).

The content of ellagic acid was determined after adding 2 g of sample to a volumetric flask, adding distilled water, hydrochloric acid and ethanol thereto, heating the mixture in a water bath at elevated temperature for 1 hour, and then performing centrifugation. C18 column (Zorbax Eclipse XDB C18, 4.6×150 mm, 5 μm) was used as the stationary phase. The mobile phase was a mixture of 5 mM potassium dihydrogen orthophosphate (pH 2.5) and acetonitrile (84:16, v:v). Flow rate was 0.43 mL/min. Measurement was made at 370 nm using a UV detector.

The content of 4-n-butylresorcinol was determined after adding 2 g of sample to a volumetric flask, adding methanol thereto, performing ultrasonication for 1 hour, and then performing centrifugation.

The content of 4-n-butylresorcinol was analyzed by HPLC. C18 column (Zorbax Eclipse XDB C18, 4.6×150 mm, 5 μm) was used as the stationary phase. The mobile phase was a mixture of 0.1% acetic acid in water and methanol (20:80, v:v). Flow rate was 0.42 mL/min. Measurement was made at 280 nm using a UV detector.

Using a calibration curve prepared using standard ellagic acid and 4-n-butylresorcinol, the content of ellagic acid and 4-n-butylresorcinol in the sample was calculated. The result is shown in [Table 1].

TABLE 1
Analysis of active ingredients in sumac (Rhus javanica L.) extract,
fermented extract and purification product

| | Content (%) | | |
|---|---|---|---|
| | Concentrated extract | Fermented | Purification |
| Active ingredients | (ethanol) | concentrate | product |
| Ellagic acid | 0.24 | 5.48 | 26.78 |
| 4-n-Butylresorcinol | — | 1.96 | 32.45 |

### Example 3

Preparation of Molecularly Encapsulated Fermented Sumac (Rhus javanica L.) Extract

The crystals obtained in <Example 2> are water-insoluble particles. They were molecularly encapsulated to prepare them into a stable, water-soluble form.

5 g of hydroxypropyl-β-cyclodextrin (HPβCD) was completely dissolved in 4 g of water at 60° C. to prepare a water-HPβCD mixture solution. Then, 5 g of the fermented sumac (Rhus javanica L.) extract fraction prepared in <Example 2> was added to the water-HPβCD mixture solution and encapsulation was carried out by cooling to room temperature at a constant cooling rate of −0.2° C./min.

In order to stabilize the capsules, the product was kept at 4° C. for 7-10 hours. The prepared molecular capsules contained 9.6% of 4-n-butylresorcinol.

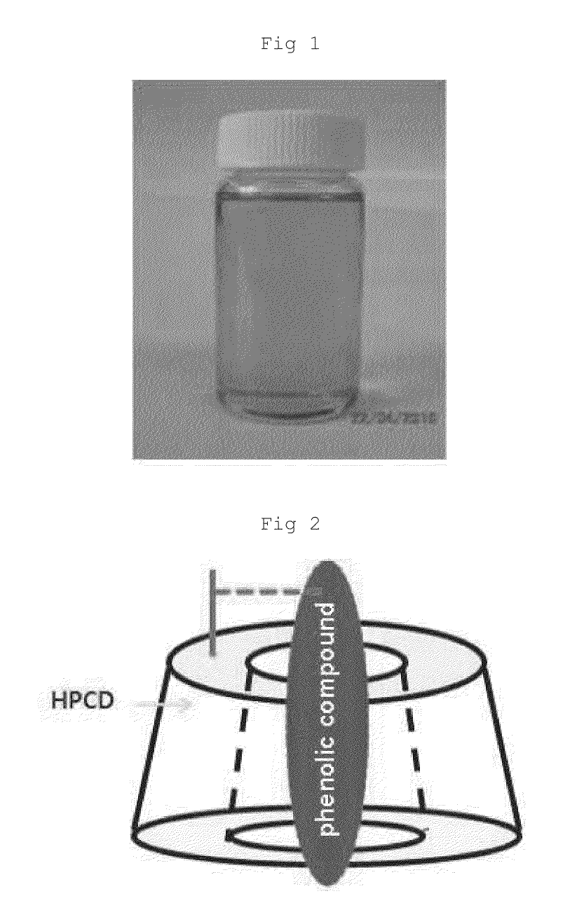

The fermented sumac (Rhus javanica L.) extract-containing molecular capsules were dissolved in water. As seen from [FIG. 1], the molecular capsules were completely dissolved in the aqueous solution.

### Example 4

Inhibition of Tyrosinase Activity by Fermented Sumac (Rhus javanica L.) Extract

Tyrosinase inhibition activity was evaluated by measuring the change in absorbance using a microplate reader.

L-Tyrosine was added to a 0.1 M sodium phosphate buffer (pH 6.8) to a concentration of 1.5 mM. After preparing the fermented extract of the present invention at 5 to 8 different concentrations using a 0.1 M sodium phosphate buffer, 1000 U/mL of tyrosinase was added to each solution. After reaction at 37° C. for 30 minutes, absorbance was measured at 475 nm using a microplate reader. The experiment was carried out 3 times and the mean value was calculated.

The same experiment was carried out using arbutin as control substance. The concentration of arbutin in the test medium was from 13.33 mM to 0.42 mM. From the concentration of arbutin when the tyrosinase activity was inhibited by 50% (IC₅₀), the arbutin concentration of the test medium was determined to be 1.555 mM (see [FIG. 4]).

The same experiment was carried out for whitenol comprising 4-n-butylresorcinol, and the result was compared with that of the fermented extract of the present invention. The concentration when the tyrosinase activity was inhibited by 50% (IC₅₀) was calculated.

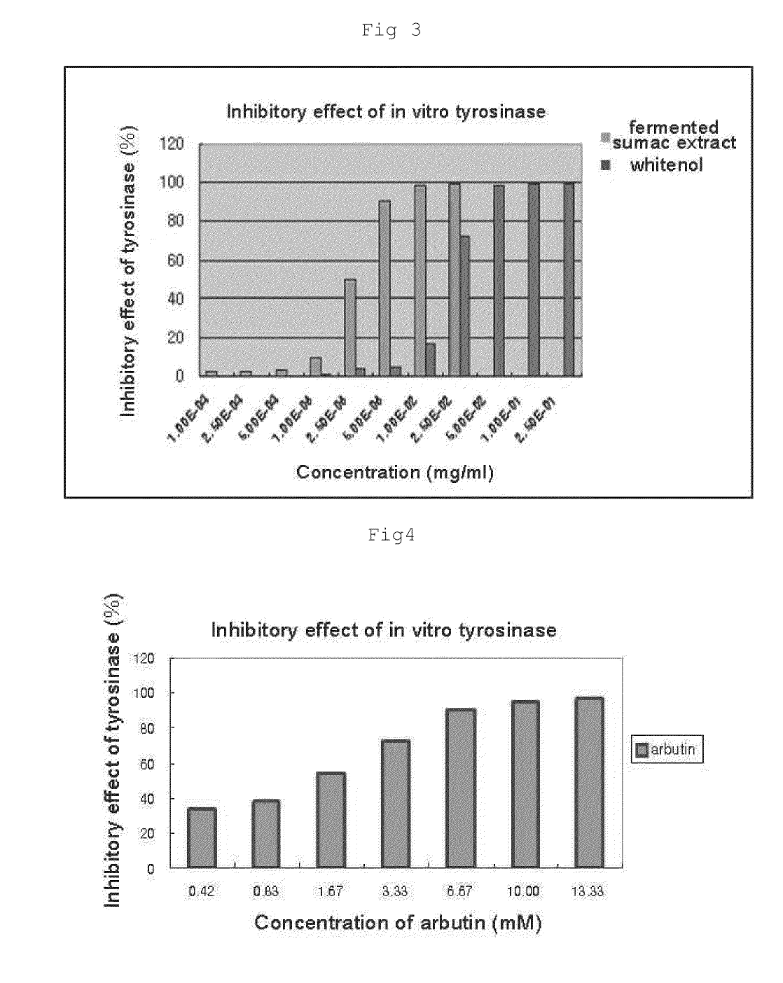

As seen from [FIG. 3], the fermented sumac (Rhus javanica L.) extract comprising 4-n-butylresorcinol showed better tyrosinase inhibition activity than whitenol. And, the IC₅₀ concentration of the fermented sumac (Rhus javanica L.) extract was 0.0025 mg/mL (4-n-butylresorcinol content=0.2395 μM), and the IC₅₀ concentration of the whitenol sample was 0.0173 mg/mL (4-n-butylresorcinol content=2.356 μM). Thus, it was confirmed that the fermented sumac (Rhus javanica L.) extract of the present invention inhibits tyrosinase activity in vitro than better whitenol. Also, as seen from [FIG. 4], the composition of the present invention inhibited tyrosinase activity much better than arbutin even at very low concentration (diluted 100 times or more).

### Example 5

Free Radical Scavenging Activity of Fermented Sumac (Rhus javanica L.) Extract

Free radical scavenging activity of the composition of the present invention was tested by modifying the method of Keun-Ha Lee, Soo-Nam Park, et al. (J. Soc. Cosmet. Scientists Korea Vol. 34, No. 1, March 2008, 25-35).

The fermented extract of differing concentration was added to 0.1 mM 1,1-diphenyl-2-picrylhydrazyl (DPPH) dissolved in ethanol. After waiting for 30 minutes, absorbance was measured at 517 nm. For comparison of activity, absorbance change was measured between the sample-free control group and the sample-containing experiment group.

1,1-Diphenyl-2-picrylhydrazyl (DPPH) used in the electron donating ability test is a stable free radical. It exhibits a maximum absorption peak at around 517 nm due to its unshared electron. When it accepts an electron or a proton, the absorbance at 517 nm decreases. Thus, a sample capable of reducing or scavenging the radical has good antioxidant and radical scavenging activities.

DPPH activity inhibition was calculated as follows.

Inhibition (%)=[(A_{Experiment}−A_{Control})/A_{Experiment}]×100

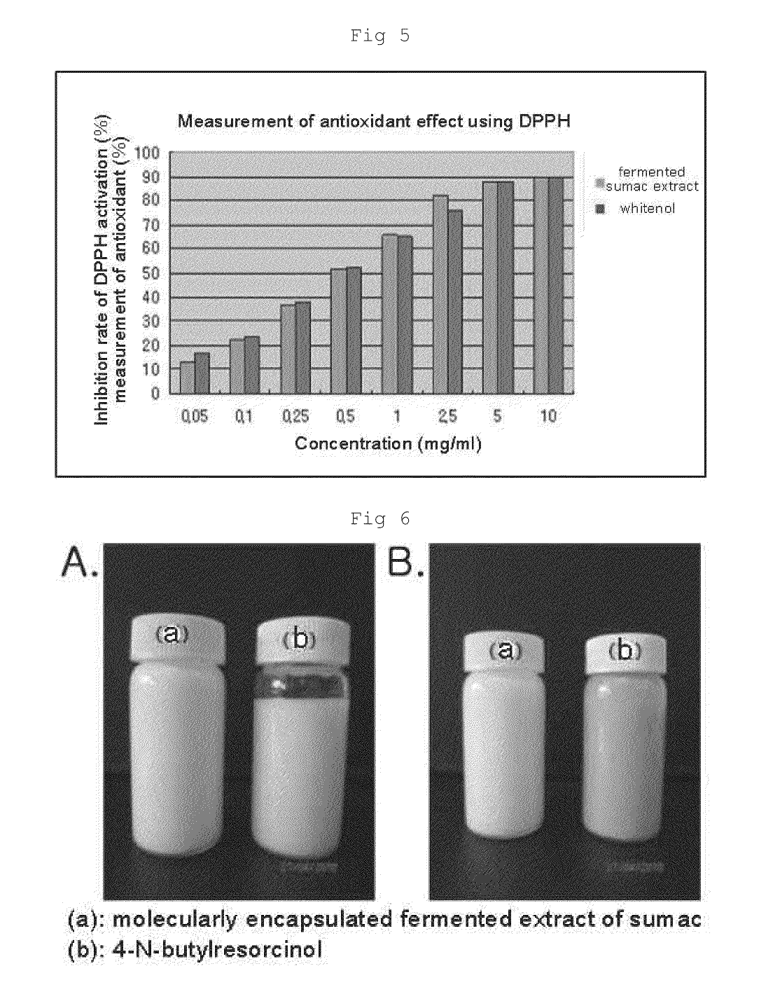

As seen from [FIG. 5], the fermented sumac (Rhus javanica L.) extract of the present invention showed better antioxidant activity than whitenol.

The content of 4-n-butylresorcinol in the fermented sumac (Rhus javanica L.) extract and the whitenol was 9.65% and 13.56%, respectively. The concentration of 4-n-butylresorcinol required to reduce the concentration of DPPH to 50% (free radical scavenging activity, FSC₅₀) was calculated.

FSC₅₀ of the fermented sumac (Rhus javanica L.) extract was 0.0466 mg/mL and that of whitenol was 0.065 mg/mL. Thus, it was confirmed that the fermented sumac (Rhus javanica L.) extract exhibits antioxidant activity although the 4-n-butylresorcinol concentration was lower than that of whitenol.

### Example 6

Stability of Molecularly Encapsulated Fermented Sumac (Rhus javanica L.) Extract Nourishing creams containing 0.95 wt % of the molecularly encapsulated fermented sumac (Rhus javanica L.) extract (0.13% of 4-n-butylresorcinol) or 4-n-butylresorcinol (0.13%) were prepared according to <Preparation Example 1>. The creams were kept at room temperature or at 45° C., and the change in color with time was observed.

As seen from [FIG. 6], after 30 days, the nourishing cream containing the molecularly encapsulated fermented sumac (Rhus javanica L.) extract showed color change neither at room temperature nor at 45° C. In contrast, the nourishing cream containing the non-molecularly encapsulated 4-n-butylresorcinol showed color change both at room temperature and at 45° C. The color change was severer when the nourishing cream had been kept at 45° C.

### Example 7

Skin Irritation of Molecularly Encapsulated Fermented Sumac (Rhus javanica L.) Extract

Nourishing creams containing 0.95 wt % of the molecularly encapsulated fermented sumac (Rhus javanica L.) extract (0.13% of 4-n-butylresorcinol) or 4-n-butylresorcinol (0.13%) were prepared according to <Preparation Example 1>. Patch test was carried out for 15 volunteers.

After applying the cream on the upper back or forearm using a patch, the patch was removed 24 to 48 hours later. After waiting for 24 hours until the temporary erythema disappeared, erythema, edema, or the like was observed and evaluated.

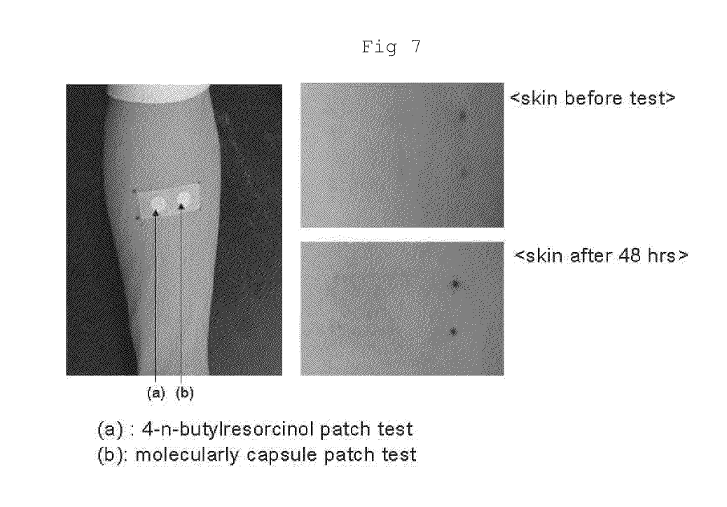

The part to which the nourishing cream containing the molecularly encapsulated fermented sumac (Rhus javanica L.) extract had been applied showed no irritation. In contrast, the part to which the nourishing cream containing 4-n-butylresorcinol had been applied showed skin irritation and erythema in 7 out of the 15 subjects. The result is shown in [FIG. 7]. Therefore, it can be seen that the molecularly encapsulated fermented sumac (Rhus javanica L.) extract according to the present invention greatly reduce irritation as compared to 4-n-butylresorcinol.

### Preparation Example 1

### Preparation of Cream

1 wt % of the composite prepared by molecularly encapsulating the fermented sumac (Rhus javanica L.) extract with a cyclodextrin derivative in <Example 3> was prepared into a nourishing cream according to a commonly used method by mixing it with 6 wt % of 1,3-butylene glycol, 4 wt % of glycerin, 5 wt % of liquid paraffin, 3 wt % of squalene, 1.5 wt % of Polysorbate 60, and purified water as balance.

1. A cosmetic composition for skin-whitening containing a composite prepared by molecularly encapsulating a fermented sumac (Rhus javanica L.) extract with a cyclodextrin derivative as an active ingredient.

2. A cosmetic composition for antioxidant containing a composite prepared by molecularly encapsulating a fermented sumac (Rhus javanica L.) extract with a cyclodextrin derivative as an active ingredient.

3. The composition of claim 1 or 2, wherein the fermented sumac (Rhus javanica L.) extract comprises 4-n-butylresorcinol in an amount of 20 to 99 wt % based on the total weight of the fermented sumac extract.

4. The composition of claim 1 or 2, wherein the cyclodextrin derivative is selected from the group consisting of 2,6-dimethyl-β-cyclodextrin, hydroxyethyl-β-cyclodextrin, hydroxyethyl-γ-cyclodextrin, hydroxypropyl-β-cyclodextrin, hydroxypropyl-γ-cyclodextrin, (2-carboxymethoxy)propyl-β-cyclodextrin or sulfobutylether-7-β-cyclodextrin.

5. The composition of claim 1 or 2, wherein the cyclodextrin derivative is hydroxyethyl-β-cyclodextrin.

6. The composition of claim 1 or 2, wherein the fermented sumac (Rhus javanica L.) extract is prepared by the method of fermenting sumac extract with Lactobacillus casei and purifying the fermentant.
